(19) United States
(12) Patent Application Publication
(10) Pub. No.: US 2020/0085864 A1
(43) Pub. Date: Mar. 19, 2020

(54) Stannous Fluoride Sugar Alcohol Solid Solutions

(71) Applicant: WhiteHill Life Sciences, LLC, Stafford, TX (US)

(72) Inventors: Dale G. Brown, Wharton, TX (US); Ira D. Hill, Austin, TX (US)

(21) Appl. No.: 16/408,515

(22) Filed: May 10, 2019

Related U.S. Application Data
(60) Provisional application No. 62/732,018, filed on Sep. 17, 2018.

(51) Int. Cl. A61K 33/16; A61K 47/26

(52) U.S. Cl. CPC A61K 33/16; A61K 47/26

(57) ABSTRACT

Stannous fluoride polyol solid solutions combine three complimentary, biochemical mechanisms attributed to Sn⁺⁺, F⁻ and Polyol, respectively to reduce growth and metabolism of Streptococcus mutans while effecting superior Bioactivity Quotients. The stannous fluoride polyol solid solution particulate compositions of the present invention comprise:
(a) stannous fluoride at between about 0.01 and about 0.8% by weight;
(b) a polyol at between about 0.1 and about 30% by weight;
(c) an astringency neutralizer at between about 0.01 and about 0.4% by weight, where the ratio of astringency neutralizer to stannous fluoride is from between about 0.01 and about 0.2;
(d) a mucoadhesive at between about 1.5 and about 70% by weight, wherein the ratio of mucoadhesive to stannous fluoride polyol is from between about 7 to 1 and about 25 to 1;
(e) a pH stabilizer, selected from the group consisting of malic, fumaric, citric acid and combinations thereof, wherein the ratio of pH stabilizer to stannous fluoride polyol is from between about 0.03 and 5 and preferably from between about 0.1 and about 3; and
   (f) optional flavorants, stabilizers, preservatives, conditioners, and oral care adjuncts.

### PRIORITY CLAIM

This application claims priority from commonly owned, copending U.S. Provisional Application No. 62/732,018, filed Sep. 17, 2018, the disclosure of which is hereby incorporated herein by reference in its entirety.

### FIELD OF THE INVENTION

The present invention relates to topical administration to oral surfaces of a mouth rinse solution resulting from the dissolution in water of solid solution particles containing stannous fluoride in sugar alcohols that combine three diverse biochemical mechanisms associated with Stannous fluoride, at its natural pH, and polyol, respectively. These three diverse biochemical mechanisms in the solid solution complement each other to reduce growth and metabolism of Streptococcus mutans, while simultaneously effecting an unprecedented Bioactivity Quotient in general oral health, caries, periodontal disease prevention and treatments.

Preferred solid solution particles of the present invention are anhydrous and glycerin-free products which include stable stannous fluoride dissolved in an appropriate sugar alcohol which also can contain other hedonic and efficacy-improving ingredients, including but not limited to an astringency neutralizer, a mucoadhesive, flavors and other compositions added to neutralize the negative taste perceptions of stannous fluoride, and a pH stabilizer for use in products and protocols responsive to oral complications associated with medications, medical treatments and/or systemic conditions.

### BACKGROUND OF THE INVENTION

Stannous fluoride (SnF₂) rinse has been used in dentistry since the 1950s as a chemical adjunct to prevent dental caries, gingivitis, and periodontitis, including the painful and slow recovery of surgery of the gums and reducing the re-infection by bacteria whether from a continuing presence or re-introduction of the organisms involved in the inflammation and destruction of the gums. Stannous fluoride is also effective for the treatment of various oral sores like aphis ulcers and yeast infections such as thrush and the terrible oral pain resulting from AIDS. It is widely considered to be the safest, most effective, over-the-counter anti-microbial since Dr. I. L. Shannon developed the stable solution of the drug in anhydrous glycerin in the late 1960's and published widely in the dental research literature.

A stannous fluoride rinse is commonly constructed by dissolving the anhydrous salt in a very dry source of glycerin, requiring long hours of high temperature “cooking” of commercial glycerin. This solution is packaged in moisture and oxygen barrier bottles, sometimes with delivery pumps or for single dose use in a pouch constructed with similarly protective and sealable films. An FDA approved concentration of the rinse entails stirring a measured quantity of the glycerin/stannous fluoride which is to be stirred by the patient into an appropriate quantity of water to yield an FDA approved OTC rinse concentration.

However, it's unacceptability for everyday use by clients at home, plus the inability of most patients to maintain long-term and effective use, often resulting in discontinuation of treatment by periodontists, general dentists and their patients, leads to severely limited use of this most effective, FDA approved, oral anti-microbial agent due to:
1. a very unpleasant and sometimes painful hedonic profile having a strong astringent property similar to green persimmons, zinc and aluminum chlorides and other salts,
2. a broad modification of multiple flavor perceptions which remains unpleasant long after the rinse is expectorated,
3. instability and pH shifts in aqueous solutions or adsorption of water by the hydroscopic nature of glycerin causing reduced shelf life after repeatedly opening the container in a humid bathroom,
4. a concurrently reduced bioactivity immediately upon dissolution in water due to moving the product outside the range of the “natural pH” of stannous fluoride, and
5. a limitation on the produce delivery procedures and formulations which can be formulated because of the high glycerin percentage in the concentrate.

One of the difficulties posed in the use of stannous fluoride in clinical dentistry has been the difficulty in stabilizing this agent in aqueous solutions or even in glycerin having a small percentage of water in it. The instability of SnF₂ is primarily due to the reactivity of the stannous ion (Sn⁺⁺). In aqueous solutions above pH 4, Sn⁺⁺ readily hydrolyzes, resulting in its precipitation from solution. In such environments, tin remains unprotected from oxidization to the inactive stannic ion (Sn⁺⁴). The first approach to eliminate these chemical changes in consumer products was to dissolve SnF₂ in an anhydrous material such as glycerin. Such gel and rinse products were further protected from loss of activity by eliminating mineral abrasives which could react with stannous fluoride. While SnF₂ in an anhydrous gel base was generally stable, it did not gain wide consumer acceptance because of (1) an unpleasant, astringency taste/feel in the mouth, (2) difficulty with using the gel with a toothbrush, and (3) the absence of abrasives. Poor patient compliance was well established with SnF₂ brushing gels and rinses in the 1980s. See Wolff, et al., JADA, Vol. 119, August 1989; 283-289 and Leverett, et al., J. Dent. Res. 63:1083-1086, 1984.

Until the present invention, it was not obvious to scientists and those skilled in the art, that some other, more acceptable, anhydrous solvent than anhydrous glycerin, could be utilized to easily manufacture many forms of oral care products, such as rinses, gels, tooth powders and dental flosses, not to mention higher concentration Rx rinses, lacquers, and other Rx forms restricted to dentist application, which exhibit even better stability and the opportunity to incorporate other ingredients which would overcome the unacceptable elements of glycerin or chelation in oral care products in general. This invention utilizes the ability of commercially available sugar alcohols to enable the manufacture and use of stannous fluoride in all forms of dental products, suitable for consumers and professional use alike.

Although not limited by economic considerations, one element of this invention is to utilize orally useful sugar alcohols having positive value of their own, such as plaque reduction and non-cariogenic effects while taking advantage of their relatively low melting points. Thus, the molten state of otherwise solid sugar alcohols such as Erythritol, Xylitol, Sorbitol, and the like, could solubilize stannous fluoride and upon cooling provide the basis for more effective, more stable, more hedonically acceptable rinses and other oral care products to be developed.

Polyhydric alcohols, or sugar alcohols as they are more commonly known, are often used as food additives that function as sweeteners, texture modifiers, crystallization modifiers, hygroscopic agents, viscosity modifiers and anticaries agents. Ly and Milgrom in Food Constituents in Oral Health 2009, pp. 134-151, reviewed sugar alcohols and dental health. Sugar alcohols/polyols are reported to have low caloric value and are non-cariogenic. The commercial execution of sweetening agents has mostly utilized sorbitol and xylitol in gums, candies, drinks, toothpastes, breath drops, etc.

Two especially preferred polyhydric alcohols used herein are Erythritol and Xylitol.

Erythritol, also described as (2R,3S)-butane-1,2,3,4-tetrol, and has the structural formula:

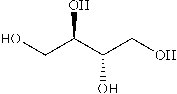

Properties and uses of Erythritol as described in Wikipedia include:
1. A poly alcohol or sugar alcohol used as a sweetener
2. A non-fermentable sugar alcohol
3. Is 60-70% as sweet as sugar
4. Is almost non-caloric
5. Does not cause tooth decay
6. Less likely to cause laxation (as compared to xylitol and maltitol
7. An FDA food additive
8. FDA recognized anticaries claim (21 CFR 101.80) (61 FR 43433, Aug. 23, 1996)
9. Industrially produced by fermentation of corn starch.

Xylitol, also described as pentane-1,2,3,4,5-pentol or 1,2,3,4,5-pentahydroxy-pentane has the structural formula:

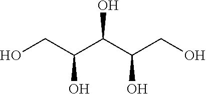

Properties and uses of Xylitol as described by Wikipedia include:
1. a poly alcohol or sugar alcohol used as a sweetener
2. a non-fermentable sugar alcohol
3. sweet as sugar with 33% fewer calories
4. reducing caries and helping remineralization
5. having more dental health benefits than other polyols
6. causing secretion of saliva
7. an FDA food additive
8. supporting the label claim, “xylitol does not promote dental cavities” and
9. industrially produced from xylose by hydrogenation or by fermentation.

Xylitol, a common sweetener is a natural, 5-carbon, sugar alcohol that cannot be fermented by bacteria. It is noncariogenic because it inhibits the formation of caries by reducing the amount of acid produced by acidogenic bacteria.

### OBJECTS OF THE INVENTION

An unmet need continues today in oral care for high-compliance, anhydrous, stable stannous fluoride compositions that can be maintained free from hydrolysis and oxidation, provided they are administered at about SnF₂'s natural pH, in order to effect optimum bioactivity in various oral care protocols that are responsive to a wide range of oral complications associated with medications, medical treatments and/or systemic conditions.

In response to this unmet need, one object of the present invention is to replace anhydrous, stannous fluoride/glycerin solutions with bioactive, anhydrous, stannous fluoride polyol solid solutions.

Another object of the invention is to improve the bioactivity quotient for stannous fluoride, oral care products by replacing (a) stannous fluoride glycerin, (b) complexed stannous fluoride, (c) chelated stannous fluoride, (d) stannous fluoride hexameta-phosphate combinations, (e) sodium fluoride, and/or (f) sodium monofluorophosphate with bioactive, stannous fluoride polyol solid solutions that include three distinctive, biochemical mechanisms, resulting in a superior Bioactivity Quotient.

A further object of the invention is to improve stannous fluoride stability, resistance to hydrolysis and oxidation, antimicrobial efficacy, Bioactivity Quotient, and patient compliance by replacing anhydrous, stannous fluoride/glycerin solutions, complexed, chelated, stannous fluoride, aqueous compositions and/or stannous fluoride hexametaphosphate compositions with bioactive, stannous fluoride polyol solid solutions with an astringency neutralizing agent.

Yet another object of the invention is to provide bioactive, stannous fluoride, solid solution rinses at the natural pH of stannous fluoride by in-situ mixing bioactive, stannous fluoride polyol solid solution compositions containing an astringency neutralizer, a mucoadhesive and a pH stabilizer with tap water prior to rinsing.

A still further object of the invention is to optimize the Bioactivity Quotient of stannous fluoride, including anticaries, remineralizing and antibacterial effects, reducing Streptococci mutans, including reducing the growth and metabolism of S. mutans, reducing plaque and gingivitis and improving bleeding index by replacing anhydrous, stannous fluoride/glycerin solutions with bioactive, anhydrous, stannous fluoride polyol solid solutions suitable for in-situ mixing with tap water, prior to rinsing.

Another object of the invention is to optimize the Bioactivity Quotient of stannous fluoride, thereby reducing and/or curing periodontal conditions, thrush, canker sores, and other microbial or viral infections of the oral cavity.

Another object of the invention is to replace stannous fluoride/glycerin solutions, complexed and/or chelated, stannous fluoride compositions and/or stannous fluoride hexametaphosphate compositions with bioactive stannous fluoride polyol solid solutions and an astringency neutralizer.

A further object of the invention is to replace anhydrous, stannous fluoride glycerin solutions, complexed and/or chelated, stannous fluoride compositions and stannous fluoride hexametaphosphate compositions with bioactive stannous fluoride polyol solid solutions and an astringency neutralizer and a pH stabilizer, in a mucoadhesive composition that enhances oral substantivity, improves patient compliance and registers a superior Bioactivity Quotient.

Specific commercial objects of the present invention include:

Providing anhydrous, bioactive, stannous fluoride polyol solid solutions in hedonically superior bases, with a pH stabilizer, a mucoadhesive and an astringency neutralizer to replace anhydrous, stannous fluoride glycerin solutions for use in bioactive, stannous fluoride polyol solid solution, in-situ rinses to be mixed with tap water.

Providing bioactive, stannous fluoride polyol solid solutions in hedonically superior bases to replace:
(a) stannous fluoride glycerin
(b) stannous fluoride (complexed and/or chelated) in current toothpastes
(c) stannous fluoride hexametaphosphate compositions
(d) NovaMin® in current toothpastes
(e) sodium fluoride or sodium monofluorophospate in current fluoride toothpastes and rinses
(f) stannous fluoride or sodium fluoride used in professionally applied, standard varnish coatings and
(g) sodium fluoride used in prophy pastes.

Providing pre-measured, bioactive stannous fluoride polyol solid solutions in hedonically superior bases containing a pH stabilizer, to be in-situ mixed with pre-measured tap water to form a bioactive, stannous fluoride rinse with a pH below 4 and a superior Bioactivity Quotient.

Providing bioactive, stannous fluoride polyol solid solution particles, suitable for compression coating combination drug/devices with superior Bioactivity Quotient.

Providing bioactive, stannous fluoride polyol solid solution particles in hedonically superior, aqueous-free toothpastes, brushing gels, prophy pastes and varnishes to replace aqueous-based, complexed stannous fluoride, stannous fluoride hexametaphosphate compositions, sodium fluoride and sodium monofluorophosphate toothpastes.

### Defined Terms

“Polyol” means a 4 carbon to 12 carbon sugar alcohol taken from the series, lacitol, isomalt, erythritol, threitol, arbitol, ribitol, galactitiol, dulcitol, allitol, iditol, mannitol, sorbitol and xylitol.

“Anhydrous” means it contains no water.

“Aqueous-free” means “not containing water” or “not dissolved in water.”

“Astringency neutralizers” means ingredients that reduce or eliminate the perception of SnF₂ astringency by hedonic sensing sites or by combinations of sensory inputs, including (1) coating the oral tissue, (2) increasing salivation to “wash out” the SnF₂ more quickly, (3) adding flavors which modify the brain's interpretation of all the inputs from a composition including those affecting taste buds, olfaction, “mouthfeel” and hedonics in general.

“Stannous fluoride polyol” means a solution of stannous fluoride in anhydrous polyol, which may be glassy solid or crystalline.

“Mucoadhesive” means an ingredient or composition of various ingredients which is retained on oral mucosal and tooth surfaces for an extended period.

“pH stabilizer” means an ingredient which maintains the stability of aqueous SnF₂ and achieves its most bioactive, “natural” or “native” pH which is nominally pH of 3.2 to 3.7.

“Stannous fluoride stability” means stannous fluoride maintained in the Sn′ valence state without appreciable degradation to stannic fluoride (Sn′) which is not bioactive. Hydrolysis and oxidation are the primary causal degradation pathways affecting stability.

“Stannous fluoride Bioactivity Quotient” means the sum of stannous and fluoride in ppm divided by the pH of the composition.

“Stannous fluoride substantivity” means the stannous fluoride moiety, especially in its “native” state is retained on the soft and hard surfaces of the oral cavity. The longer it is thus retained increases the benefits of stannous fluoride.

“Patient compliance” means the composition is sufficiently pleasant, during and after use, to motivate patients to consistently comply with the patient use instructions.

“Oral complications” means painful, diseased and/or degraded oral surfaces caused by side effects medication and/or medical treatments.

“in-situ mixed rinses” means mixed just prior to use.

“Natural pH” means ingredients soluble in water quickly adjust to a specific pH (acidic or alkaline) upon dissolution. Equilibrium is reached when the pH remains stable. This is the “natural pH”.

“pH stabilizers” means readily saliva soluble ingredients that reduce the pH of compositions of the invention introduced into the oral cavity to improve the Bioactivity Quotient of the compositions.

“Stannous fluoride complexing or chelating” means chemical forms which complex, bind, tie-up or otherwise interfere with stannous fluoride's ability to reach its “natural pH” and to react effectively to various microorganisms, enamel surfaces, tissue surfaces, taste buds, etc.

“Conditions free from oxidation and hydrolysis of stannous fluoride” means prevent moisture and air from reaching the stannous fluoride moiety and rendering it ineffective.

“Hedonically superior” means more pleasurable.

“Solid solution” a solid-state solution. A solute in a solvent. Such a mixture is considered a solution rather than a compound when the crystal structure of the solvent remains substantially unchanged by addition of the solute, and when the mixture remains in a single homogenous phase.

“Solid solution particles” means micronized particles of a “solid solution” of the solvent such as xylitol occurring in which the crystal structure of the solvent is substantially unchanged. The solid solution particles are generated by treatment of ½ inch to ¾ inch chunks with a Waring blender, and sieved through a 70 mesh screen which passes everything below 200 microns. Accordingly, the stannous fluoride/polyol micronized particles have a size range of from about 1 to about 200 microns, and preferably from about 50 to about 150 microns.

“Biochemical mechanism” means a chemical mechanism involved in vital processes occurring in living organisms.

“Complimentary” means chemical, physical or drug actions working together to accomplish an effect.

“Protocols” means guidelines, sequences or procedures to achieve a desired effect such as alleviating a specific oral complication.

“Bioactivity Quotient for solid solutions of the present invention” means:

$$\frac{\begin{matrix}[ Fluoride level ( PPM ) + Stannous Level ( PPM ) ] \times \\ Polyol Level \% by weight\end{matrix}}{pH}$$

“At-risk patients” means those patients whose general oral health and/or oral complications can exacerbate serious, sometimes life-threatening, medical conditions in other parts of the body.

### BRIEF DESCRIPTION OF THE DRAWINGS

FIGS. 1 to 5 schematically illustrate the invention. Solid solution particles containing SnF₂ and polyol, combine to control S. mutans adhesion, metabolism and growth when topically administered to oral surfaces in a mucoadhesive that gels upon contact with oral surfaces. Saliva flow dissolves the mucoadhesive gel, releasing Sn⁺⁺, F⁻ and polyol to carry out three complimentary, biochemical mechanisms of action for controlling S. mutans.

FIG. 1 schematically illustrates solid solution particulates of Sn⁺⁺, F⁻ and polyol.

FIG. 2 schematically illustrates the solid particulates shown in FIG. 1 dispersed in an anhydrous mucoadhesive.

FIG. 3 schematically illustrates the mucoadhesive with particulates shown in FIG. 2 having formed a gel upon contacting oral surfaces.

FIG. 4 schematically illustrates the gel with particulates shown in FIG. 3 dissolving, as well as the dissolving of particulate releasing Sn⁺⁺, F⁻ and xylitol onto the oral surface with S. mutans to pursue their complimentary, biochemical mechanisms of action directed to S. mutans.

FIG. 5 schematically shows the S. mutans depicted in FIG. 4 after the Sn⁺⁺, F⁻ and polyol have effected adhesion, growth and metabolism of S. mutans.

### SUMMARY OF THE INVENTION

The present invention relates to solid solution particles containing stannous fluoride polyol that combine three diverse biochemical mechanisms associated with SnF₂ at its natural pH, and polyol, respectively. Topically administered to oral surfaces, the Sn⁺⁺, F⁻ and polyol complement each other to reduce growth and metabolism of Streptococcus mutans, reduce or eliminate periodontal infection, thrush, canker sores, and other oral inflammations, while simultaneously effecting an unprecedented Bioactivity Quotient.

In a preferred embodiment of the invention, anhydrous, solid solution particles of the present invention are mixed with an astringency neutralizer, a mucoadhesive and a pH stabilizer for use in products and protocols responsive to oral complications associated with medications, medical treatments and/or systemic conditions.

The solid solution particles of the present invention, suspended in anhydrous compositions containing a mucoadhesive such as polydimethylsiloxane emulsified in a poloxamer form a gel upon contacting oral surfaces. The SnF₂ and polyol solid solution particles in the gel come in direct contact with oral surfaces as the saliva soluble mucoadhesive gel is solubilized by saliva flow, thereby effecting these complimentary, biochemical modes of action on S. mutans.

A further embodiment of the present invention relates to stable, high compliance, bioactive compositions comprising a mixture of a solid solution of stannous fluoride polyol, with astringency neutralizers, mucoadhesives and pH stabilizers suitable for use in a broad range of stannous fluoride, oral care products that are more responsive to various oral complications than current, fluoride, and oral care products.

Stannous fluoride/polyol solid solution technology of the present invention was developed to protect all oral surfaces of “at-risk” patients, with a long-lasting, high-compliance, bioactive, stannous fluoride/polyol solid solution by regularly brushing, rinsing and flossing with stannous fluoride/polyol solid solution-containing:
toothpastes, brushing gels, prophy pastes and varnishes
rinses to be in-situ mixed with tap water and
dental flosses or dental tapes.

Rx Stannous fluoride/polyol toothpastes, rinses, dental flosses and dental tapes are prescribed in various protocols for protecting oral surfaces of “at-risk” patients using medications, undergoing medical treatment and/or indicating systemic conditions.

The efficacy of stannous fluoride in toothpastes, rinses, etc., with regard to caries, hypersensitivity, periodontitis, thrush, and other microbial infections, has been well established in the literature.

Bioactive, anhydrous, stannous fluoride polyol solid solution powders of the present invention, with astringency neutralizers and pH stabilizers such as fumaric, citric and malic acid when in-situ mixed with tap water, produce a bioactive rinse with optimum antibacterial activity at about the natural pH of stannous fluoride with minimal oxidation when promptly used after in-situ mixing.

Bioactive, anhydrous, stannous fluoride polyol solid solution compositions of the present invention are preferred over:
1. stannous fluoride/glycerin solutions
2. chelated or complexed stannous fluoride used in toothpastes
3. stannous fluoride hexametaphosphate compositions
4. sodium fluoride used in toothpastes, rinses, gels and varnishes and
5. sodium mono fluorophosphates compositions used in gels and rinses.

The basis for these preferences for bioactive, stannous fluoride polyol solid solution compositions include:
Superior stability with respect to oxidation and hydrolyzation,
Superior Bioactivity Quotient,
Superior substantivity,
Achieving natural pH rapidly when in-situ mixed with tap water, and
Superior patient compliance.

The control of Streptococcus mutans exhibited by the stannous fluoride polyol solid solution compositions of the present invention confirms that this solid solution combination of bioactive stannous fluoride and polyol may result in a synergistic effect for controlling Streptococcus mutans levels.

The optimum pH for this synergistic effect on Streptococcus mutans may not be the “natural pH of stannous fluoride, i.e., 3.2 to 3.5 but rather slightly higher, but under 4.0, where the weight ratio of SnF₂ to polyol ranges from between about 0.005 to 0.10 and about 0.03 to 0.08.

Preferred embodiments of the invention include the following:

1. A stannous fluoride polyol solid solution particulate compositions comprising:

(a) stannous fluoride at between about 0.01 and about 0.8% by weight;

(b) a polyol at between about 0.1 and about 30% by weight;

(c) an astringency neutralizer at between about 0.01 and about 0.4% by weight, where the ratio of astringency neutralizer to stannous fluoride is from between about 0.01 and about 0.2;

(d) a mucoadhesive at between about 1.5 and about 70% by weight, wherein the ratio of mucoadhesive to stannous fluoride polyol is from between about 7 to 1 and about 25 to 1;

(e) a pH stabilizer, selected from the group consisting of malic, fumaric, citric acid and combinations thereof, wherein the ratio of pH stabilizer to stannous fluoride polyol is from between about 0.03 and 5 and preferably from between about 0.1 and about 3; and

(f) optional flavorants, stabilizers, preservatives, conditioners, and oral care adjuncts.

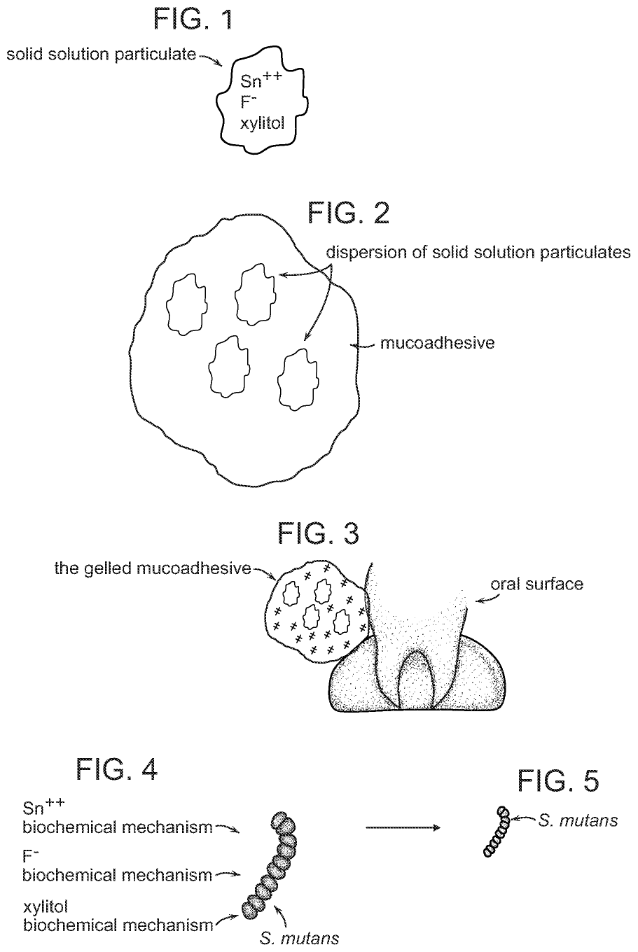

2. The stannous fluoride polyol solid solution particulate of Embodiment 1, wherein the polyol is selected from the group consisting of erythritol, xylitol, isomalt, maltitol, sorbitol, and combinations thereof.
3. The stannous fluoride polyol solid solution particulate of Embodiment 1, which combine three complimentary, biochemical mechanisms attributed to Sn⁺⁺, F⁻ and polyol to reduce growth and metabolism of Streptococcus mutans, growth and metabolism, as well as curing of microbial or viral infections producing thrush, periodontitis, canker sores, and other oral diseases, while effecting superior Bioactivity Quotients.
4. The stannous fluoride polyol solid solution particulates of Embodiment 1, further suspended in an anhydrous oral care composition selected from the group consisting of toothpastes, brushing gels, prophy pastes, varnishes, muco-adherent films, and combinations thereof, which reduce growth and metabolism of Streptococcus mutans, growth and metabolism of organisms, resulting in the curing or mitigation of microbial or viral infections producing thrush, periodontitis, canker sores, and other oral diseases, while effecting Bioactivity Quotients of at least about 7000.
5. The stannous fluoride polyol solid solution particulates of Embodiment 1, suitable for in-situ mixing with tap water to reduce growth and metabolism of Streptococcus mutans while effecting Bioactivity Quotients of at least about 800.
6. The stannous fluoride polyol solid solution particulates of Embodiment 1, suitable for coating dental devices, which combine three complimentary, biochemical mechanisms attributed to: Sn⁺⁺, F⁻ and polyol to reduce growth and metabolism of Streptococcus mutans, reducing or eliminating infections producing thrush, periodontitis, canker sores, and other oral diseases, while effecting Bioactivity Quotients of at least about 1700, by working the stannous fluoride polyol particulates between tooth surfaces that cannot be reached by brushing or rinsing.
7. Anhydrous compositions containing stannous fluoride polyol solid solution particulates, an astringency neutralizer, a mucoadhesive and a pH stabilizer, suitable for oral care applications, which indicate superior stability, Bioactivity Quotient, substantivity and patient compliance.
8. Stable, anhydrous, oral care compositions containing stannous fluoride polyol solid solution particulates, an astringency neutralizer, a mucoadhesive and a pH stabilizer, wherein:
upon in-situ mixing with tap water, produce a rinse, at a pH under 4, that is responsive to oral complications associated with medications, medical treatments and/or systemic conditions including caries, plaque buildup, gingivitis, periodontitis, mucositis, stomatitis, thrush, hypersensitivity, hyposalivation and combinations thereof.
   9. Stable, anhydrous, oral care compositions containing stannous fluoride polyol solid solution particulates suspended in a mixture containing an astringency neutralizer, a mucoadhesive and a pH stabilizer, and mixed with another oral care composition selected from the group consisting of toothpaste, brushing gel, varnish, prophy paste and combinations thereof, having a superior Bioactivity Quotient that is responsive to oral complications associated with medications, medical treatments including chemotherapy and radiation, and systemic conditions including caries, plaque buildup, gingivitis, mucositis, periodontitis, stomatitis, thrush, hypersensitivity, hyposalivation and combinations thereof.
   10. Stable, oral care compositions containing stannous fluoride polyol solid solution particulates, an astringency neutralizer, a mucoadhesive and a pH stabilizer for use in coated dental devices selected from the group consisting of multifilament dental floss, monofilament dental tape, proxy brushes, dental flossers and dental picks, and suitable for releasing said stannous fluoride polyol solid solution particulates interproximally, in response to oral complications associated with medications, medical treatments and/or systemic conditions including caries, plaque buildup, gingivitis, periodontitis, mucositis, stomatitis, thrush, hypersensitivitiy, hyposalivation and combinations thereof.
   11. In-situ rinse compositions, according to Embodiment 8, wherein said pH stabilizer is selected from the group consisting of fumaric acid, malic acid, citric acid and combinations thereof.
   12. In-situ rinse compositions, according to Embodiment 8, wherein said mucoadhesive is selected from the group consisting of anhydrous emulsions of polydimethylsiloxane as the discontinuous phase and poloxamer surfactants as the continuous phase.
   13. In-situ rinse compositions, according to Embodiment 8, wherein said astringency neutralizer is selected from the group consisting of spilanthes extract, Jambu resin extract, synthetic spilanthol, pellitorine, Echinacea extract and combinations thereof.
   14. In-situ rinse compositions, according to Embodiment 8, wherein the pH of said rinse during rinsing is about the natural pH of stannous fluoride.
   15. In-situ rinse compositions, according to Embodiment 8, wherein said rinse is substantially free from oxidation during in-situ mixing.
   16. In-situ rinse compositions, according to Embodiment 8, wherein the fluoride level in said stannous fluoride polyol solid solution powder is above 0.1% stannous fluoride and said rinse is classified as an Rx prescription drug.
   17. Bioactive, anhydrous toothpaste compositions containing stannous fluoride polyol solid solution particulate, according to Embodiment 9, wherein the fluoride level is above 0.1% and said toothpaste is classified as an Rx prescription drug.
   18. Bioactive, anhydrous prophy paste compositions containing stannous fluoride polyol solid solution particulates, according to Embodiment 9, wherein the fluoride level is above 0.1% and said prophy paste is classified as an Rx prescription drug.
   19. Bioactive, anhydrous varnish compositions containing stannous fluoride polyol solid solution particulates, according to Embodiment 9, wherein said fluoride level is above 0.1% and said varnish is classified as an Rx prescription drug.
   20. A method for manufacturing anhydrous, stannous fluoride polyol solid solution particulates under conditions free from oxidation and hydrolysis of stannous fluoride comprising:
(a) heating the polyol to its melting temperature;
(b) followed by the addition, with stirring, of anhydrous stannous fluoride powder;
(c) followed by crystallization of said mixture; and
(d) followed by micronizing the mixture to appropriate particle size particulates under conditions free from hydrolyzing and oxidizing stannous fluoride.
   21. A method for treating oral complications associated with medications, medical treatment, systemic conditions, and the like, comprising topically applying to the oral cavity, an oral care composition containing a mixture of bioactive, stannous fluoride polyol solid solution particulates with an astringency neutralizer, a mucoadhesive and a pH stabilizer, effecting a superior Bioactivity Quotient, as defined in Embodiment 1.
   22. A method for treating oral complications, according to Embodiment 21, wherein said topically administered, oral care composition is selected from the group consisting of anhydrous toothpaste, brushing gel, varnish, prophy paste and coated dental devices.
   23. A method for treating oral complications, according to Embodiment 21, wherein said topically administered, oral care compositions include bioactive, stannous fluoride polyol solid solution powder, in-situ mixed with tap water prior to rinsing and expectorating.
   24. A method for treating oral complications according to Embodiment 21, where in the polyol is selected from the group consisting of erythritol, xylitol, isomalt, maltitol, sorbitol, and combinations thereof.
   25. A method for treating oral complications selected from the group consisting of gingivitis, periodontitis, yeast infections, thrush, and other oral diseases related to microbes, comprising topically applying to the oral cavity, an oral care composition containing a mixture of bioactive, stannous fluoride polyol solid solution particulates with an astringency neutralizer, a mucoadhesive and a pH stabilizer, effecting a superior Bioactivity Quotient, as defined in Embodiment 1.
   26. A method for treating oral complications, according to Embodiment 25, wherein said topically administered, oral care composition is selected from the group consisting of anhydrous toothpaste, brushing gel, varnish, prophy paste and coated dental devices.
   27. A method for treating oral complications, according to Embodiment 25, wherein said topically administered, oral care compositions include bioactive, stannous fluoride polyol solid solution powder, in-situ mixed with tap water prior to rinsing and expectorating.
   28. A method for treating oral complications according to Embodiment 25, where in the polyol is selected from the group consisting of erythritol, xylitol, isomalt, maltitol, sorbitol, and combinations thereof.
   29. A method for controlling S. mutans in the oral cavity, as schematically illustrated in FIGS. 1 through 5 of the Drawings, using the stannous fluoride polyol solid solution particulate composition of Embodiment 1.

It will be appreciated by those persons having ordinary skill in the art(s) to which the present invention relates that any of the features described herein in respect of any particular embodiment and/or embodiment of the present invention can be combined with one or more of any of the other features of any other embodiments and/or embodiments of the present invention described herein, with modifications as appropriate to ensure compatibility of the combinations. Such combinations are considered to be part of the present invention contemplated by this disclosure.

It is to be understood that both the foregoing general description and the following detailed description are exemplary and explanatory only and are not restrictive of the invention as claimed. Other embodiments will be apparent to those skilled in the art from consideration of the specification and practice of the invention disclosed herein.

### DETAILED DESCRIPTION OF THE INVENTION

Bioactive, stannous fluoride polyol solid solutions in various oral compositions of the present invention outperform, on the basis of Bioactivity Quotient:
(a) stannous fluoride glycerin, solution compositions
(b) complexed or chelated, stannous fluoride compositions
(c) stannous fluoride hexametaphosphate compositions
(d) sodium fluoride compositions and/or
(e) sodium monofluorophosphate compositions. See Bioactivity Quotient data reported below.

Stannous fluoride polyol solid solutions are formed by dissolving stannous fluoride powder in heated polyol. Upon cooling, the solution forms stable, anhydrous crystals. The Bioactivity Quotient for stannous fluoride polyol solid solutions used in rinses and toothpastes, is superior to the Bioactivity Quotient for other fluoride toothpastes and rinses, as shown in Tables I and II below:

TABLE I
Stannous Fluoride Xylitol Solid Solution Toothpaste
Compared to Leading Fluoride Toothpaste Brands

| | | Fluoride | Stannous | | Xylitol | | |
|---|---|---|---|---|---|---|---|
| Fluoride | Regulatory | Level | Level | | Level | Bioactivity | Astringency |
| Toothpaste Brand | Status | (PPM) | (PPM) | pH | (% by wt) | Quotient * | Neutralizer |
| Stannous fluoride | Rx | 1650 | 5158 | 3.8 | 7.0 | 12541 | Yes |
| xylitol | | | | | | | |
| Stannous fluoride | OTC | 1100 | 3440 | 3.8 | 6.0 | 7168 | Yes |
| xylitol | | | | | | | |
| PRO- | OTC | 1100 | 3440 | 6.6 | — | 688 | No |
| HEALTH ®** | | | | | | | astringency |
| SENSODYNE ®** | OTC | 1100 | 3440 | 7.1 | — | 639 | No |
| Stannous fluoride | | | | | | | astringency |
| TOTAL ® | OTC | 1100 | — | 6.8 | — | 162 | N.A. |
| Sodium fluoride | | | | | | | |
| CREST ® | OTC | 1100 | — | 6.6 | — | 167 | N.A. |
| Sodium fluoride | | | | | | | |
| ClinPro ™ | Rx | 1100 | — | 6.6 | — | 758 | N.A. |
| Sodium Fluoride | | | | | | | |

* Bioactivity Quotient for stannous fluoride 1 POLYOL solid solution toothpaste=

[Fluoride level(PPM)+Stannous Level(PPM)]×Polyol level in %/pH
Note: Should there be no polyol present, the Bioactivity Quotient is based on fluoride level (PPM) and stannous fluoride (PPM) divided by the pH. The higher the Bioactivity Quotient—the more effective against Streptococcus mutans.

TABLE II
Stannous Fluoride Xylitol Solid Solution, in-situ Rinse Compared to Leading Rinse Brands

| | | Fluoride | Stannous | | Xylitol | | |
|---|---|---|---|---|---|---|---|
| Fluoride | Regulatory | Level | Level | | Level | Bioactivity | Astringency |
| Rinse Brand | Status | (PPM) | (PPM) | pH | (% by wt.) | Quotient * | Neutralizer |
| Stannous fluoride | Rx | 374 | 1136 | 3.5 | 4.0 | 1725 | Yes |
| xylitol | | | | | | | |
| (0.15% SnF₂) | | | | | | | |
| Stannous fluoride | OTC | 243 | 758 | 3.6 | 3.0 | 834 | Yes |
| xylitol | | | | | | | |
| (0.1% SnF₂) | | | | | | | |
| Act ® | OTC | 226 | — | 6.6 | — | 34 | No |
| (0.05% NaF) | | | | | | | astringency |
| LISTERINE ® | OTC | 90 | — | 3.78 | — | 26 | No |
| (0.02% NaF) | | | | | | | astringency |
| PRO- | OTC | 90 | — | 4.7 | — | 21 | No |
| HEALTH ® | | | | | | | astringency |
| (0.02% NaF) | | | | | | | |
| Perio-Med ® | Rx | 243 | 758 | 4.64 | — | 216 | No |
| (0.1% SnF₂) | | | | | | | |
| Gel-Kam ® | Rx | 243 | 758 | 4.64 | — | 216 | No |
| (0.1% SnF₂) | | | | | | | |

* Bioactivity Quotient for stannous fluoride xylitol solid solution rinse=

$$\frac{\begin{matrix}[ Fluoride level ( PPM ) + Stannous Level ( PPM ) ] \times \\ Xylitol Level \% by weight\end{matrix}}{pH}$$

Note: Where there is no xylitol present, the Bioactivity Quotient is based on fluoride level (PPM) and stannous fluoride (PPM) divided by the pH.

The stannous fluoride polyol solid solution toothpaste, rinse and dental devices of the present invention combine three complimentary, biochemical mechanisms by delivering a stannous fluoride polyol solid solution onto oral surfaces of “at-risk” patients. This solid solution outperforms other fluorides in Bioactivity Quotient and in thoroughness of protecting all surfaces, in response to medications, medical treatments and/or systemic conditions.

The bioactive, stannous fluoride polyol solid solutions of the present invention are preferred over other fluoride compositions on the basis of:
stability,
substantivity,
maintaining natural pH,
Bioactivity Quotient, and
patient compliance.

### Comparison to Prior Art Stannous Fluoride Glycerin Solutions:

Using anhydrous, bioactive, stannous fluoride polyol solid solutions of the present invention as the source for stannous fluoride in certain oral care compositions obviates certain difficulties inherent in working with stannous fluoride glycerin solutions, i.e.,
Stability issues associated with the extensive heating required to reduce the glycerin water content and minimize the risk of hydrolysis.
Bulk issues, i.e., a 55 gallon drum of stannous fluoride glycerin solution is equivalent on available fluoride basis to about a 5 gallon container of bioactive, stannous fluoride polyol solid solution particulates.
Bioactivity issues, where stannous fluoride glycerin solutions diluted in tap water generally fail to reach “natural” stannous fluoride pH levels during rinsing, i.e., about 3.2 to 3.5. See Bioactivity Quotient data presented in Table II.
Compliance issues associated with astringency of stannous fluoride glycerin that discourages regular patient use.
Substantial energy savings in melting and solubilizing stannous fluoride in polyol versus the 180° C. heating and lengthy cool-down time lost in glycerin/SnF₂ solutions.
   Comparison to Complexed and/or Chelated, Stannous Fluoride and Stannous Fluoride Hexamethaphosphate, Aqueous-Based, Toothpaste Compositions:

Anhydrous, stannous fluoride/polyol solid solution particulate toothpastes indicate superior Bioactivity Quotients compared to complexed, chelated, stannous fluoride-based, aqueous toothpastes and stannous fluoride hexametaphosphate toothpaste. This superior Bioactivity Quotient, reported in Table I above is most evident upon comparing antibacterial, antiplaque, antigingivitis and antifungal properties of the various toothpastes.

Bioactive, stannous fluoride polyol solid solutions are not as vulnerable to oxidation, nor to hydrolysis, and show improved Bioactivity Quotient at stannous fluoride natural pH, when formulated in an aqueous-free toothpaste, compared to complexed, chelated stannous fluoride in aqueous-based toothpastes or stannous fluoride hexametaphosphate toothpaste. Specifically, aqueous-based, stannous fluoride toothpastes require the stannous fluoride be complexed to minimize hydrolysis. This results in increased pH, as shown in Table I above.

This resistance to hydrolysis in complexed, aqueous, stannous fluoride compositions is accompanied by a dramatic reduction in antibacterial activity is confirmed in Tables I and II.

In summary, chelated stannous fluoride used in water-based toothpastes, while indicating minimal stannous fluoride hydrolysis and acceptable stability, fails to provide bioactive, stannous ions at about the natural pH of stannous fluoride resulting in low Bioactivity Quotients as reported in Table I above.

In contrast, the bioactive, stannous fluoride polyol solid solution compositions of the present invention, used in aqueous-free toothpaste, maintain stannous ions without complexing, achieve stannous fluoride, natural pH levels, assuring an optimum Bioactivity Quotient, as reported above. Note: An astringency neutralizer is required for these compositions to overcome the astringency associated with bioactive Complexed stannous fluoride used in aqueous-based toothpastes interferes with providing stannous ions to oral surfaces in an optimum, bioavailable form at the natural pH of stannous fluoride. Such complexing of the stannous ion:
1. reduces antibacterial activity and antiplaque effect of stannous fluoride compared to bioactive, stannous fluoride polyol solid solutions of the present invention used in aqueous-free toothpaste;
2. reduces the control of Streptococcus mutans compared to bioactive, stannous fluoride xylitol solid solutions of the present invention used in aqueous-free toothpaste; and
3. adversely affects oral surface, antibacterial properties (retention of tin in plaque) compared to bioactive, stannous fluoride polyol solid solution particulates of the present invention used in aqueous-free toothpaste. See Bioactivity Quotient results reported in Table I above.

Complexing Stannous Fluoride Used in Aqueous-Based Toothpastes Interferes with Providing Stannous Fluoride at Its Natural pH (about 3.2), which is required for:
1. optimizing Bioactivity Quotient. This includes antibacterial and antiplaque properties; and
2. optimizing antibacterial activity. This includes retention of tin in plaque, resulting in improved control of Streptococcus mutans.

Complexing stannous fluoride for use in aqueous-based toothpastes ties up substantial amounts of stannous ions to a great extent, reducing dramatically the astringency characteristic of stannous fluoride. This astringency property of stannous fluoride/glycerin (where the stannous moiety is uncomplexed) has plagued the commercial acceptance of stannous fluoride/glycerin products from the early 1950s to the present, based on noncompliance. This complexed stannous fluoride used in aqueous-based toothpastes obviates the “compliance problems” associated with uncomplexed stannous fluoride/glycerin compositions.

In contrast, bioactive, stannous fluoride polyol solid solution particulate compositions of the invention, containing a mucoadhesive and an astringency neutralizer are preferred by a wide margin over stannous fluoride glycerin compositions on the basis of mouthfeel, cooling, refreshing taste and absence of astringency.

Bioactive, stannous fluoride polyol solid solutions with astringency neutralizers, formulated into aqueous-free toothpaste and in-situ rinses, indicate exceptional hedonics and consumer preference scores superior to aqueous-based, complexed, stannous fluoride toothpastes and stannous fluoride/glycerin rinses diluted with tap water. These consumer preferences are generally based on:
1. a longer lasting mouthfeel,
2. enhanced saliva flow,
3. longer lasting, cool, refreshing flavor, and
4. an immediate tingling sensation that is associated with cleaning action.

Complexed stannous fluoride used in aqueous-based toothpastes compared to bioactive, stannous fluoride polyol solid solutions used in aqueous-free toothpastes, suggests complexed stannous fluoride not only represents a compromise in toothpaste Bioactivity Quotient but also complexed stannous fluoride fails to effect the S. mutans control indicated by stannous fluoride xylitol solid solution toothpastes of the present invention.

Comparing the stannous levels in plaque of patients using complexed stannous fluoride in aqueous-based toothpastes with the stannous levels in plaque of patients using bioactive, stannous fluoride polyol solid solution particulates in aqueous-free toothpastes of the present invention shows a substantial advantage for bioactive, stannous fluoride polyol solid solution, toothpaste compositions of the present invention, with respect to:
stannous levels in plaque,
reduced levels of Streptococcus mutans in the oral cavity, and
enhanced hardness of enamel.

The emergence of bioactive, stannous fluoride polyol solid solution particulates suitable for use in aqueous-free toothpastes offer major advances in:
stannous fluoride stability,
stannous fluoride Bioactivity Quotient,
stannous fluoride substantivity, and
stannous fluoride superior patient compliance;
   as discussed above. This suggests that various, bioactive, stannous fluoride polyol solid solution particulates and aqueous-free toothpastes also be considered as replacements for sodium fluoride and sodium monofluorophosphate, aqueous-based toothpastes, which generally fall short in Bioactivity Quotient, as shown above.

Similarly, aqueous-based, sodium fluoride rinses also lack the bioactivity and substantivity properties exhibited by bioactive, stannous fluoride xylitol solid solution powder compositions suitable for in-situ mixing with tap water, prior to rinsing. Fluoride rinses that fall short in antibacterial activity include shortfalls in controlling Streptococcus mutans and establishing fluoride substantivity on oral surfaces required for optimal oral protection. See Bioactivity Quotient reported in Table II above.

The preferred bioactive, stannous fluoride polyol solid solution compositions of the present invention comprise:
1. stannous fluoride at between about 0.01 and about 0.8% by weight
2. polyol at between about 0.1 and about 30% by weight
3. an astringency neutralizer at between about 0.01 and about 0.4% by weight, where the ratio of astringency neutralizer to stannous fluoride is from between about 0.01 and about 0.2
4. a mucoadhesive at between about 1.5 and about 70% by weight, wherein the ratio of mucoadhesive to stannous fluoride polyol is from between about 7 to 1 and about 25 to 1
5. a pH stabilizer, selected from the group consisting of malic, fumaric, citric acid and combinations thereof, wherein the ratio of pH stabilizer to stannous fluoride polyol is from between about 0.03 and 5 and preferably from between about 0.1 and about 3 and
6. flavorants, stabilizers, preservatives, conditioners, various oral care adjuncts, etc., as required, provided these ingredients are compatible with the stability, bioactivity, substantivity and compliance properties of the present invention.

Anhydrous, stannous fluoride polyol solid solution compositions of the present invention are available as OTC or Rx prescription drugs, depending on the level of stannous fluoride present therein, i.e.,
Anhydrous, stannous fluoride polyol solid solution particulate compositions also containing astringency neutralizer(s), mucoadhesive(s), pH stabilizer(s), flavors, conditioners and preservatives. These compositions are available in bulk for use in aqueous-free toothpastes, brushing gels, prophy pastes and varnishes.
Anhydrous, stannous fluoride polyol solid solution, particulate compositions for coating various interproximal delivery means, such as dental flosses, dental tapes, proxy brushes, flossers, etc.
Anhydrous, stannous fluoride polyol solid solution, particulate compositions for use in rinses in-situ mixed with tap water.

Stannous fluoride polyol solid solution compositions of the present invention are used in various oral care products to protect all oral surfaces, in response to the oral complications associated with:
medications,
medical treatments, and
systemic conditions.
   experienced by “at-risk” patients, especially those with “dry mouth” symptoms, e.g., from drugs, chemotherapy, or radiation treatments for cancer.

These stannous fluoride polyol solid solution products of the present invention, with superior Bioactivity Quotients, replace traditional oral care products including those described in Tables I and II above.

Examples 1 through 23 below describe stannous fluoride polyol solid solution particulates containing toothpastes, in-situ rinses, dental floss, dental tape, prophy paste, varnishes, as well as various oral care PROTOCOLS containing these stannous fluoride polyol solid solution products for use by “at-risk” patients (1) preparing for medical treatment, (2) during medical treatment, and (3) after medical treatment.

### EXAMPLES

### Example 1

Anhydrous, bioactive, stannous fluoride xylitol solid solution particulates were prepared as follows:

Low water content xylitol (preferably less than 0.5% water) was melted and stirred, followed by the addition of anhydrous, stannous fluoride, solid powder for 30 minutes to one hour with dry nitrogen above the molten liquid. The liquid was allowed to cool below the melting point with a small amount of previously crystallized stannous fluoride xylitol solid solution added as seed crystals. Once crystallization was complete, the resulting solids were crushed, pulverized and screened to produce bioactive solid solution particulates suitable for manufacturing oral care compositions, including rinses, gels, toothpastes, prophy pastes, varnishes, coating for dental devices, etc.

Astringency neutralizers, mucoadhesives and pH stabilizers can be added to the solid solutions prior to formulating oral care compositions.

### Example 2

A 250 mL glass beaker was fitted with an overhead stirrer. Dry xylitol powder (0.1% moisture), 98.5 gm, was added to the beaker and heated on a hot plate at 100° C. with stirring until a clear liquid is present. Stannous fluoride powder at 1.5 gm was then added over 30 seconds with continued stirring. After 30 minutes, no visual particles of stannous fluoride were evident. The solution was then poured out on a metal sheet and allowed to crystallize over 12 hours. The resulting solid mass was then broken up into small chunks and added to a Waring blender. The chunks were reduced in size during 3 minutes of high speed blender action. The powder was then screened through a 70 mesh screen to give a white powder suitable for preparing powdered stannous fluoride xylitol solid solution particulate suitable for formulating anhydrous gel, toothpaste, in-situ rinse, etc., compositions.

### Example 3

A powdered rinse concentrate of the invention, suitable for in-situ mixing with tap water, was prepared by mixing several powders. The first powder, A, was taken from Example 2. The 1.5% stannous fluoride xylitol solid solution powder, 0.99 gm, was weighed out. A second powder, B, was prepared comprising mucoadhesive components. ULTRAMULSION® 810/12.5 (an emulsion of poloxamer 338 stirred with polydimethylsiloxane at 12,500 cs) was prepared by melting and stirring for 30 minutes. The mucoadhesive melt was cooled and solidified. Chunks were broken off and pulverized with a Waring blender at high speed over 3 minutes. The powder, B, was screened through a 50 mesh screen. A portion of the ULTRAMULSION® 810/12.5 powder at 18.6 grams was weighed out into a 250 mL glass beaker. Fine powdered sucralose, 1.77 gm, was added to the beaker with stirring by spatula. A blend of vanillamint flavor and spilanthes extract (astringency neutralizer), 2.64 gm, was added to the beaker also with manual stirring. Benzoic acid powder (pH accelerator) at 0.015 gm was added to the beaker with stirring resulting in a flowable powder. A portion of the blend of powders A and B in the beaker at 0.296 gm was added to a plastic weighing dish and the stannous fluoride/xylitol powder (Powder A), 0.99 gm was added. The blend of powders was stirred to give a homogeneous mixture. The 1.286 gm of the resulting powder blend was added to a glass vial with cap. Tap water at 15 mL was added to the vial. The cap was affixed and the mixture shaken for 10 seconds. The uniform suspension/emulsion was dispensed for oral rinsing. A pleasant tasting rinse was taken into the mouth and swished for 30 seconds. The experience was devoid of metallic, astringency taste experienced with commercial stannous fluoride/glycerin rinses, such as those described in Table II. A pleasant after taste lasted for 5 minutes, largely without the annoying metallic, astringent, residual taste.

### Example 4

Powder A from Example 3 at 1.485 gm was weighed out into a plastic dish. Powder B from Example 3 at 0.429 gm was added to the plastic weighing dish and mixed with a spatula. The blend of powders A and B was stirred to give a homogeneous mixture. 1.914 gm of the powder blend was added to a glass vial with cap. Tap water at 15 mL was added to the vial. The cap was affixed and the mixture shaken for 10 seconds. The uniform suspension/emulsion from the vial was dispensed into the oral cavity, followed by rinsing for 30 seconds. The resulting pleasant taste was devoid of metallic, astringent taste experienced with commercial stannous fluoride/glycerin rinses, such as described in Table II. A pleasant after taste lasted for 5 minutes, largely without the annoying, metallic, astringent, residual taste. A pH of this formula in water after 1 minute was 4.69.

### Example 5

A powdered rinse concentrate of the invention, suitable for in-situ mixing with tap water, was prepared by mixing several powders. The first powder, A, comprised a solid solution of stannous fluoride xylitol solid solution comprising 96.7% by wt. xylitol and 3.3% by wt. stannous fluoride. A second powder, B, comprised mucoadhesive emulsion described in Example 3 at 80.8% by wt, vanillamint P flavor at 10%, sucralose at 7.7% and astringency neutralizer at 0.15% by wt. An in-situ powder rinse comprising 0.682 g of powder B and 0.444 g of powder A was blended and in-situ mixed with 15 mL tap water. The pH of the in-situ mix at 30 seconds was 4.68 and the pH of the in-situ mix at 60 seconds was 4.68. Upon the addition of 10 mg of fumaric acid to the blend of powders A and B, followed by in-situ mixing with 15 ml of tap water, the pH of the in-situ rinse at 30 seconds was 3.886 and the pH of the in-situ rinse at 60 seconds was 3.998.

### Example 6

Powder A from Example 3, 1.485 gm, was weighed out into a plastic dish. Powder B from Example 3, 0.429 gm, was added to the plastic weighing dish and mixed with a spatula. Fumaric acid powder, 0.010 gm, was added. The blend of powders was stirred to give a homogeneous mixture. Then 1.914 gm powder blend was added to a glass vial with cap. Tap water, 15 mL, was added to the vial. The cap was affixed and the mixture shaken for 10 seconds. The uniform suspension/emulsion was dispensed for oral rinsing. A pleasant tasting rinse was taken into the mouth and rinsed for 30 seconds. The experience was devoid of metallic taste experienced with commercial stannous fluoride/glycerin rinses. A pleasant after taste lasted for 5 minutes, largely without the annoying metallic, astringent, residual taste. A pH of this formula in water after 1 minute was 3.95.

### Example 7

To a 2 Liter stainless steel beaker, fitted with an overhead stirrer, was added 475.1 gm of anhydrous glycerin. 12 gm of hydroxypropylcellulose was added with stirring over 1 minute and continued stirring for 10 minutes. Polyethylene glycol 400, 180 gm, was added to the beaker with stirring. ULTRAMULSION® L-1220/2.5 mm PDMS, 20%, was added with continued stirring. A powder of stannous fluoride, 8%, in xylitol, 66 gm, was added with stirring. Sucralose, 0.6 gm, was added. Flavors, 12.3 gm, were added with continued stirring. Pumice, F, 336 gm, was added over 3 minutes with stirring. Additional pumice FF, 168 gm, was added over 3 minutes with stirring. TiO₂, 12 gm, was added with stirring over 1 minute. The mixture was transferred to a Hobart mixer for continued blending. Aerosil 200, 15 gm was added over 3 minutes and blending continued for 10 minutes. The thickened prophy paste was transferred to 2 oz jars for use in polishing teeth.

### Example 8

A 16 quart, stainless steel container was fitted with an overhead stirrer and a hot plate. Poloxamer 407, 1398 gm, was added with stirring and heating until a molten liquid was formed at 90° C. Polydimethylsiloxane, 1000 Cs, 138 gm, was added over 1 minute and stirring continued for 15 minutes. Pluracare L-1220, 90 gm, was added to the heated beaker with continued stirring for 3 minutes. Stearyl alcohol flakes, 375 gm, were added over 5 minutes with stirring. Microwax ML-445, 207 gm was added over 7 minutes with continued stirring. Polyethylene 8000, 312 gm, was added over 10 minutes to the stainless container. Sident 10, 120 gm, was added to the vessel while stirring insoluble saccharin, 60 gm, was added while stirring was continued. Flavors, 206 gm, were added with stirring. The vessel was fitted with a homogenizer and 1.5% stannous fluoride/xylitol powder, 165 gm, was added over 2 minutes at 90° C. while the contents were homogenized. The thoroughly blended contents were added to floss manufacturing line applicator tank. Coating multifilament nylon yarn with this batter at 90 mg per yard gave a pleasant tasting, flossing experience delivering stannous fluoride between the teeth. Metallic, astringent taste was minimal.

Examples 9-23 are presented in the following Tables:

TABLE III
Stannous Fluoride Polyol Solid Solution, Powder Rinses

| | Stannous | (% by | Astringency | | pH |
|---|---|---|---|---|---|
| | Fluoride | wt.) | Neutralizer | Mucoadhesive | stabilizer |
| Example # | (% by wt.) | Polyol | (% by wt.) | (% by wt.) | (% by wt.) |
| 9 | 1.5% | 98.5% | Spilanthes | ULTRAMULSION ® | Fumaric |
| | | xylitol | extract | poloxamer | acid |
| | | | 0.6% | 407/2.5 mm CS | 0.9% |
| | | | | (20%) PDMS | |
| | | | | 35.2% | |
| 10 | 1.5% | 98.5% | Spilanthes | ULTRAMULSION ® | Citric acid |
| | | xylitol | extract | poloxamer 338/2.5 mm | 0.5% |
| | | | 0.4% | CS (10%) | |
| | | | | PDMS | |
| | | | | 47% | |
| 11 | 1.5% | 98.5% | Spilanthes | CMC | Malic acid |
| | | xylitol | extract | 12% | 0.3% |
| | | | 0.45% | | |
| 12 | Rx Stannous | 96.7% | Spilanthes | Hydyroxypropyl | Fumaric |
| | Fluoride | xylitol | extract | Cellulose | acid |
| | 3.3% | | 0.6% | 3.8% | 0.75% |
| 13 | Rx Stannous | 92% | Spilanthes | Mixture of | Citric acid |
| | Fluoride | xylitol | extract | ULTRAMULSION ® | 0.8% |
| | 8% | | 0.5% | poloxamer 407/2.5 mm | |
| | | | | CS (20%) | |
| | | | | PDMS 30% & | |
| | | | | hydroxypropyl | |
| | | | | cellulose | |
| | | | | 3.4% | |

TABLE IV
Stannous Fluoride Xylitol Solid Solution Particulates in Aqueous-Free Toothpastes

| | Stannous | (% by | Astringency | | pH |
|---|---|---|---|---|---|
| | Fluoride | wt.) | Neutralizer | Mucoadhesive | stabilizer |
| Example # | (% by wt.) | Polyol | (% by wt.) | (% by wt.) | (% by wt.) |
| 14 | 0.454% | 6.2% | Jambu | ULTRAMULSION ® | Malic acid |
| | | xylitol | 0.8% | poloxamer 407/2.5 mm | 0.6% |
| | | | | CS PDMS (20%) | |
| | | | | 39% | |
| 15 | 0.454% | 6.2% | Jambu | ULTRAMULSION ® | Citric acid |
| | | xylitol | 1.0% | poloxamer 338/2.5 mm | 0.5% |
| | | | | CS PDMS (10%) | |
| | | | | 42% | |
| 16 | 0.454% | 6.2% | Spilanthes | Poloxamer 407 (30%) | Fumaric |
| | | xylitol | extract | hydroxylpropylcellulose | acid |
| | | | 0.4% | 3.4% | 0.8% |
| 17 | 0.681% | 5.8% | Spilanthes | Poloxamer 338(35%) | Fumaric |
| | | xylitol | Extract | CMC (2.8%) | acid |
| | | | 0.5% | | 1.0% |
| 18 | 0.681% | 5.8% | Spilanthes | ULTRAMULSION ® | Malic acid |
| | | xylitol | Extract | poloxamer 407/2.5 mm | 1.1% |
| | | | 0.75% | CS PDMS (20%) | |
| | | | | 29% | |
| 19 | 0.908% | 7.7% | Spilanthes | ULTRAMULSION ® | Citric Acid |
| | | xylitol | Extract | poloxamer 338/2.5 mm | 1.2% |
| | | | 0.9% | CS (10%) | |
| | | | | 41% | |
| 20 | 0.908% | 7.7% | Jambu | Poloxamer 338 (36%) | Fumaric |
| | | xylitol | 1.0% | Hydroxypropylcellulose | Acid |
| | | | | 3.2% | 1.1% |

TABLE V
Bioactive, Stannous Fluoride Xylitol Solid Solution Particulates suitable for
Brushing Gels, Prophy Paste, Tooth Powders and Dental Flosses

| | Stannous | (% by | Astringency | | pH |
|---|---|---|---|---|---|
| | Fluoride | wt.) | Neutralizer | Mucoadhesive | stabilizer |
| Example # | (% by wt.) | Polyol | (% by wt.) | (% by wt.) | (% by wt.) |
| 21 | 1.5% | 98.5% | Jambu | ULTRAMULSION ® | Malic Acid |
| | | Xylitol | 1.1% | poloxamer 407/2.5 mm | 0.75% |
| | | | | CS PDMS | |
| | | | | (20%) | |
| | | | | 40% | |
| 22 | 8% | 92% | Spilanthes | Poloxamer 407 | Citric acid |
| | | xylitol | Extract | 38% | 0.9% |
| | | | 0.9% | | |
| 23 | 3.3% | 96.7% | Spilanthes | ULTRAMULSION ® | Fumaric |
| | | xylitol | Extract | poloxamer 338/2.5 mm | Acid |
| | | | 0.38% | CS PDMS | 0.75% |
| | | | | (10%) | |
| | | | | 41% | |

### Example 24

A 16 quart, stainless steel container was fitted with an overhead stirrer and a hot plate. Poloxamer 407, 2787 gm, was added with stirring and heating until a molten liquid was formed at 90° C. Polydimethylsiloxane, 1000 Cs, 276 gm, was added over 1 minute and stirring continued for 15 minutes. Pluracare L-1220, 180 gm, was added to the heated beaker with continued stirring for 3 minutes. Stearyl alcohol flakes, 661 gm, were added over 5 minutes with stirring. Microwax ML-445, 372 gm was added over 7 minutes with continued stirring. Polyethylene 8000, 558 gm, was added over 10 minutes to the stainless container. Sident 10, 240 gm, was added to the vessel while stirring insoluble saccharin, 120 gm, and sucralose, 9 gm, was added while stirring was continued. Flavors, 276 gm, were added with stirring. The vessel was fitted with a homogenizer and 8% stannous fluoride/xylitol powder, 420 gm, was added over 2 minutes at 90° C. while the contents were homogenized. Stannous fluoride powder, 100.8 gm, finely divided with less than 100 micron size, was added and the thoroughly blended contents were added to floss manufacturing line applicator tank. Coating PTFE tape with this batter at 70 mg per yard gave a pleasant tasting, flossing experience delivering stannous fluoride between the teeth. Metallic, astringent taste was minimal.

### Example 25

A powdered rinse concentrate of the invention, suitable for in-situ mixing with tap water, was prepared. A stainless container was fitted with an overhead stirrer while 29.7 grams of ULTRAMULSION® 810/PDMS (10 percent by weight of a mixture of PDMS 2.5 mm CS (66%) and AntiFoam AF1500 (34%) flakes were added. The vessel was heated to 90° C. and 4.13 grams of flavor added. Sucralose powder, 2.98 grams silica, 2.32 grams and fumaric acid powder, 0.35 grams were added with stirring for 3 minutes. A powder was prepared, 60.57 grams, formed from grinding and screening crystallized solids from a molten mixture of erythritol, 56.18 grams, 3.03 grams of xylitol, 1.36 grams of stannous fluoride. The powder was added to the stainless container with stirring for 4 minutes. The viscous melt was spread out on a stainless sheet and cooled at room temperature overnight. The lumpy mass was ground in a Waring blender and screened to give a white flowable powder. This powder, 1.126 grams was added to 0.5 ounce glass vial with a poly-seal cap. Fifteen mL of tap water was added and the cap attached. The vial was shaken for 10 seconds to give a homogeneous suspension. The contents of the vial was used to rinse the mouth. A pleasant refreshing rinse was perceived with minimal metallic taste.

### Example 26

A 250 mL glass beaker was fitted with an overhead stirrer. Dry erythritol powder (0.1% moisture), 98.5 gm, was added to the beaker and heated on a hot plate at 100° C. with stirring until a clear liquid is present. Stannous fluoride powder at 1.5 gm was then added over 30 seconds with continued stirring. After 30 minutes, no visual particles of stannous fluoride were evident. The solution was then poured out on a metal sheet and allowed to crystallize over 1 hour. The resulting solid mass was then broken up into small chunks and added to a Waring blender. The chunks were reduced in size during 3 minutes of high speed blender action. The powder was then screened through a 70 mesh screen to give a white powder suitable for preparing powdered stannous fluoride erythritol solid solution particulate suitable for formulating anhydrous gel, toothpaste, in-situ rinse, etc., compositions.

The following Examples further illustrate various oral care products of the invention:

TABLE VI
Attribute Comparison of Fluoride Rinses

| | Fluoride Type | | |
|---|---|---|---|
| | | | Stannous Fluoride |
| | Sodium | Stannous Fluoride | Polyol Solid |
| | Fluoride | Glycerin Solution | Solution Powder |
| Diluent | Water/ | in-situ mixed with | in-situ mixed with |
| | alcohol | tap water | tap water |
| pH | 5.6 | 4.6 | 3.2-3.4 |
| Antibacterial | None | S. mutans | S. mutans |
| Properties | | | |
| Bioactivity | 45 | 216 | 834 |
| Astringency | None | none | Spilanthes extract |
| Neutralizer | | | |
| Mucoadhesive | None | none | ULTRAMULSION ® |
| Tin level in | None | Limited | Up to 40% by wt. |
| plaque | | | |
| Compliance | Good | poor | Good |

TABLE VII
Attribute Comparison of Fluoride Toothpastes

| | Fluoride Type | | |
|---|---|---|---|
| | Aqueous- | | Non-aqueous, |
| | based | Aqueous-based, | Stannous Fluoride |
| | Sodium | Stannous Fluoride, | Polyol Solid Solution |
| | Fluoride | Complexed | Particulates Powder |
| pH | 5.6 | 6.2 | 3.2-3.4 |
| Antibacterial | None | S. mutans | S. mutans |
| Properties | | | |
| Bioactivity | None | 688 | 7168 |
| Astringency | None | none | Spilanthes extract |
| Neutralizer | | | |
| Mucoadhesive | None | none | ULTRAMULSION ® |
| Tin level in | None | limited | Up to 40% by wt. |
| plaque | | | |

### Polyol/SnF₂ Stability Data

A test protocol was established to compare stannous ion stability in a controlled temperature and humidity environment. A one gallon glass jar was fitted with a support platform to lift a 2 gram sample of 0.63% SnF₂ in the polyol being tested. A high humidity environment was selected at 75% using saturated sodium chloride in water at 40° C. The samples were stored under these conditions for 10 days and then assayed for stannous ion using periodate/iodide titration with starch indicator.

TABLE VIII

| | Concentration | Residual | Crystallization | |
|---|---|---|---|---|
| Polyol | of SnF₂ | stannous ion | ease | |
| glycerin | 0.63% | 26.1% | NA | |
| Xylitol | 0.63% | 34.8% | 6-12 | hours |
| sorbitol | 0.63% | 61.6% | 1 | hour |
| erythritol | 0.63% | 68.6% | 2-4 | minutes |
| Erythritol/xylitol(95:5) | 0.63% | 7.4% | 30-40 | minutes |

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

All references and other documents cited above are hereby incorporated herein by reference in their entirety.

What is claimed is:

1. Stannous fluoride polyol solid solution particulate compositions comprising:
(a) stannous fluoride at between about 0.01 and about 0.8% by weight;
(b) a polyol at between about 0.1 and about 30% by weight, wherein the polyol is selected from the group consisting of erythritol, xylitol, isomalt, maltitol, sorbitol, and combinations thereof;
(c) an astringency neutralizer at between about 0.01 and about 0.4% by weight, where the ratio of astringency neutralizer to stannous fluoride is from between about 0.01 and about 0.2;
(d) a mucoadhesive at between about 1.5 and about 70% by weight, wherein the ratio of mucoadhesive to stannous fluoride polyol is from between about 7 to 1 and about 25 to 1;
(e) a pH stabilizer, selected from the group consisting of malic, fumaric, citric acid and combinations thereof, wherein the ratio of pH stabilizer to stannous fluoride polyol is from between about 0.03 and 5 and preferably from between about 0.1 and about 3; and
(f) optional flavorants, stabilizers, preservatives, conditioners, and oral care adjuncts.

2. The stannous fluoride polyol solid solution particulates of claim 1, further suspended in an oral care composition selected from the group consisting of toothpastes, brushing gels, prophy pastes, varnishes, muco-adherent films, and combinations thereof, which reduce growth and metabolism of Streptococcus mutans, growth and metabolism of organisms, resulting in the curing or mitigation of microbial or viral infections producing thrush, periodontitis, canker sores, and other oral diseases.

3. The stannous fluoride polyol solid solution particulates of claim 1, wherein upon in-situ mixing with tap water, produce a rinse composition at a pH under 4, that is responsive to oral complications associated with medications, medical treatments and/or systemic conditions including caries, plaque buildup, gingivitis, periodontitis, mucositis, stomatitis, thrush, hypersensitivity, hyposalivation and combinations thereof.

4. The rinse composition of claim 3, wherein said pH stabilizer is selected from the group consisting of fumaric acid, malic acid, citric acid and combinations thereof.

5. The rinse composition of claim 3, wherein said mucoadhesive is selected from the group consisting of anhydrous emulsions of polydimethylsiloxane as the discontinuous phase and poloxamer surfactants as the continuous phase.

6. The rinse composition of claim 3, wherein said astringency neutralizer is selected from the group consisting of spilanthes extract, Jambu resin extract, synthetic spilanthol, pellitorine, Echinacea extract and combinations thereof.

7. The rinse composition of claim 3, wherein the pH of said rinse during rinsing is about the natural pH of stannous fluoride.

8. The rinse composition of claim 3, wherein said rinse is substantially free from oxidation during in-situ mixing.

9. The rinse composition of claim 3, wherein the fluoride level in said stannous fluoride polyol solid solution powder is above 0.1% stannous fluoride and said rinse is classified as a prescription drug.

10. A method for manufacturing anhydrous, stannous fluoride polyol solid solution particulates under conditions free from oxidation and hydrolysis of stannous fluoride comprising the steps of:
(a) heating the polyol to its melting temperature;
(b) adding anhydrous stannous fluoride powder to the melted polyol from step (a);
(c) crystallizing the mixture formed in step (b); and
(d) micronizing the mixture to form particulates under conditions free from hydrolyzing and oxidizing of the stannous fluoride.

11. A method for treating oral complications comprising topically applying to the oral cavity, an oral care composition containing a mixture of bioactive, stannous fluoride polyol solid solution particulates, with an astringency neutralizer, a mucoadhesive and a pH stabilizer, as defined in claim 1.

12. The method for treating oral complications according to claim 11, wherein the oral complication is selected from the group consisting of gingivitis, periodontitis, yeast infections, thrush, and other oral diseases related to microbes.

13. The method for treating oral complications according to claim 11, wherein said complications are associated with medications, medical treatment, systemic conditions, and the like.

14. The method for treating oral complications according to claim 11, wherein said topically administered, oral care composition is selected from the group consisting of anhydrous toothpaste, brushing gel, varnish, prophy paste and coated dental devices.

15. The method for treating oral complications according to claim 11, wherein said topically administered, oral care compositions include bioactive, stannous fluoride polyol solid solution powder, is in-situ mixed with tap water to form a rinse, followed by oral rinsing and expectorating.

16. The method for treating oral complications according to claim 15, wherein the polyol is selected from the group consisting of erythritol, xylitol, isomalt, maltitol, sorbitol, and combinations thereof.

17. The method for treating oral complications according to claim 15, wherein the pH of said rinse during rinsing is about the natural pH of stannous fluoride.

18. The method for treating oral complications according to claim 15, wherein said rinse is substantially free from oxidation during in-situ mixing.

19. The method for treating oral complications according to claim 15, wherein the fluoride level in said stannous fluoride polyol solid solution powder is above 0.1% stannous fluoride and said rinse is classified as a prescription drug.
